(19) United States
(12) Patent Application Publication
(10) Pub. No.: US 2008/0199880 A1
(43) Pub. Date: Aug. 21, 2008

(54) SPIN ARRAY METHOD

(76) Inventors: MARC D. PORTER, AMES, IA (US); JEREMY D. DRISKELL, AMES, IA (US); ROBERT J. LIPERT, AMES, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 11/677,464

(22) Filed: Feb. 21, 2007

(51) Int. Cl. G01N 33/53

(52) U.S. Cl. 435/7.1

(57) ABSTRACT

An improvement in heterogeneous immunoassays to significantly reduce assay time, from as much as 50% up to 90% of what used to be typical assay times. The improvement involves rotating the captured substrate during incubation times for antigen capture and during incubation times for sample labeling.

### BACKGROUND OF THE INVENTION

Immunoassay tests hold an important niche in human and veterinary medicine, and in bioterrorism prevention. Even with the success and widespread use of these tests, improvements in sensitivity, specificity, speed, cost, and throughput remain critical needs. This invention seeks to provide improvements in the speed and sensitivity offered by many of the methodologies employed in heterogeneous assays.

Heterogeneous immunoassays require the delivery of antigen to a solid capture substrate, and typically rely on diffusion as the mode of mass transport. Though easily implemented, diffusion limited mass transfer often results in long incubation times because large biological targets (e.g., proteins, viruses, and bacteria) have small diffusion coefficients. This limitation is amplified for sandwich-type assays since a tagged antibody is needed in order to identify and quantify the surface-bound antigen.

Various approaches have been investigated to increase the flux of the antigen or label as a means to reduce incubation time, capitalizing on the fact that antibody-antigen binding is often limited by mass transport rather than by binding kinetics (i.e., recognition rate). Electric fields, for example, have been used to drive the transport of charged species in DNA hybridization assays and in heterogeneous immunoassays. The combination of superparamagnetic labels and magnetic fields have also been shown to be an effective pathway to increase flux.

Typical heterogeneous immunoassays involve two steps that are significantly time limited. The first of the two steps is sample incubation in order to bind or capture the antigen to the substrate as illustrated FIG. 1. The second step involves attaching labels to the antigen bound to the substrate in order to allow detection. In FIG. 1, this overall process is illustrated sample 10, is incubated with the antigen 12 to form a sandwich composite 14. Thereafter, the sandwich composite 14 is reacted with a detection label 16 to form the detection composite 18. The two limiting steps involve the sample incubation period 20 and the label incubation period 22. In a typical process such as for example surface-enhanced Raman spectroscopy there is a twelve hour incubation for sample incubation 20 and a twelve hour sample incubation for the label incubation 22. This results in twenty four hours of incubation time for each assay! This extremely lengthy time period means significantly decreased economics for running these assays. There are a variety of ways that have been explored in the past in order to decrease significantly test times and as well, to enhance detection. For example, in the past, rotation of an immunoassay has been used to enhance detection. (see Huet, “A heterogeneous immunoassay performed on a rotating carbon disk electrode with electrocatalytic detection”, J. of Immunological Methods, 135:33-41 (1990)). It is important to note however that Huet is not addressing decreased assaying time, but rather enhanced detection. Put another way, Huet involves spinning or rotation of the composite sample 18 during detection, saying nothing of what happens during the already completed typical 24 hour period of sample incubation and label incubation, 20, 22.

Utilizing the technique of this invention as hereinafter described, it is possible to reduce typical times to perform surface-enhanced Raman spectroscopy from 24 hours to 25 minutes! This is demonstrated in the example below.

Accordingly, it is a primary objective of the present invention to improve a process of performing heterogeneous immunoassays by dramatically cutting the time for each assay. The method and means of accomplishing this primary objective as well as others will become apparent from the detailed description of the inventions which follows hereinafter.

### BRIEF SUMMARY OF THE INVENTION

An improvement in heterogeneous immunoassays to significantly reduce assay time, from as much as 50% up to 90% of what used to be typical assay times. The improvement involves rotating the captured substrate during incubation times for antigen capture and during incubation times for sample labeling.

### BRIEF DESCRIPTION OF THE DRAWINGS

FIG. 1 shows a typical set of steps in a heterogeneous immunoassay as primarily limited by incubation times for antigen and label attachment to the assay sandwich.

FIG. 2 is a schematic of the process of this invention as applied Raman spectroscopy to create the extrinsic Raman label (ERL).

FIG. 3 shows the Raman spectra for a blank solution and for varied concentrations of rabbit IgG.

FIGS. 4A and 4B show response curves for detection of rabbit IgG demonstrating the affect of substrate rotation.

FIG. 5 shows the effect of rotation on IgG (diluted in phosphate buffered saline (PBS)) bound to the capture substrate.

FIG. 6 shows the effective rotation and incubation during the labeling step 22 of FIG. 1.

FIG. 7 shows measured intensities versus (IgG) for each substrate in plot form and shows the results of the assay in the example and those of a control assay (no rotation).

FIG. 8 shows assay intensities for comparison purposes of rabbit IgG and goat serum matrix, for stagnant conditions and rotation at 800 rpm for 15 minutes during incubation periods.

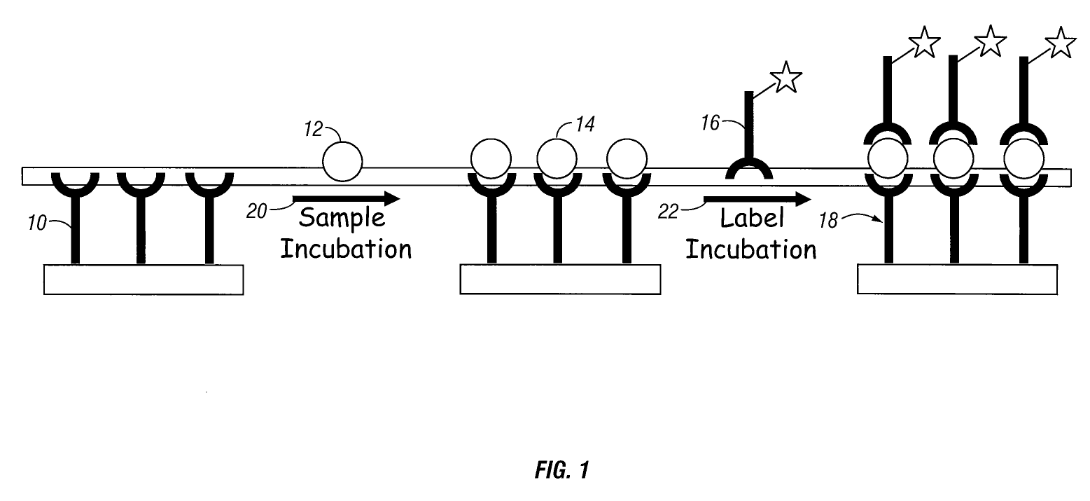

### DETAILED DESCRIPTION OF THE PREFERRED EMBODIMENT

These inventors recently described their improvement of capture substrate rotation as a means to enhance flux in heterogeneous assays (Driskell, J. D.; Kwarta, K. M.; Lipert, R. J.; Porter, M. D.; Vorwald, A.; Neill, J. D.; Ridpath, J. F. J. Virol. Methods 2006, 138, 160-169). This article is of course not prior art against the invention as is mentioned here for completeness. That paper examined the effectiveness of substrate rotation in the reduction of the time required for the antigen (i.e., virus) binding step. It also enumerated the captured viruses in a label free format by using force microscopy (AFM), noting that AFM is more readily applied in imaging objects the size of viruses but not of the proteins featured in the work herein. Moreover, the paper showed that the accumulation of bound antigen, represented by its surface concentration Γₐ, is given by (Driskell, J. D.; Kwarta, K. M.; Lipert, R. J.; Porter, M. D.; Vorwald, A.; Neill, J. D.; Ridpath, J. F. J. Virol. Methods 2006, 138, 160-169)

$${Γ}_{α} = \frac{2}{{π}^{1 / 2}} {D}^{1 / 2} {C}_{b} {t}^{1 / 2} + \frac{{D}^{2 / 3} {C}_{b}}{1.61 {V}^{1 / 6}} t {ω}^{1 / 2} \qquad ( 1 )$$

where D is the antigen diffusion coefficient, C_{b} is the bulk concentration of antigen, t is the incubation time, V is the kinematic viscosity of the solution, and ω is the rotation rate. The first term on the right hand side of the equation represents the contribution of diffusional mass transfer, whereas the second term defines the role of substrate rotation on hydrodynamically-accelerated mass transfer. There are three assumptions central to the derivation of this equation. (Driskell, J. D.; Kwarta, K. M.; Lipert, R. J.; Porter, M. D.; Vorwald, A.; Neill, J. D.; Ridpath, J. F. J. Virol. Methods 2006, 138, 160-169). First, the reactant solution concentration is independent of binding. Second, the binding sites at the surface do not saturate. Third, the recognition reaction is fast compared to the delivery of reactant. Equation 1 explicitly describes how antigen binding can be manipulated by varying t and, more importantly, ω.

There are a few precedents for use of rotation in sandwich-type heterogeneous immunoassays. For example, Huet, supra, used rotation-controlled flux to devise an assay that was independent of sample volume. Other laboratories employed rotation in the amperometric detection step of an enzymatically generated redox probe. (Wijayawardhana, C. A.; Purushothama, S.; Cousino, M. A.; Halsall, H. B.; Heineman, W. R. J. Electroanal. Chem. 1999, 468, 2-8; Salinas, E., Torriero, A. A. J.; Sanz, M. I.; Battaglini, F.; Raba, J. Talanta 2005, 66, 92-102; Messina, G. A.; Torriero, A. A. J.; DeVito, I. E.; and Olsina, R. A.; Raba, J. Anal. Biochem. 2005, 337, 195-202). To our knowledge, this invention is the first to describe a rotation-based method designed to reduce both the antigen 20 and label binding 22 times. While the specific results and example herein described pertain to the use of a nanoparticle labeling scheme which exploits surface enhanced Raman scattering (SERS), the overall strategy can be applied to virtually any type of heterogeneous assay (e.g., scintillation counting, chemiluminescence, electrochemical, enzymatic methods, surface plasmon resonance, quantum dots, and microcantilevers.

As a proving ground for the merits of rotation in a sandwich immunoassay, the below described example uses a SERS-based labeling/readout scheme previously developed by the inventors. This scheme uses extrinsic Raman labels (ERLs) to identify and quantify antigens in a sandwich immunoassay format. ERLs consist of gold nanoparticles that are coated with a layer of an intrinsically strong Raman scatterer that acts as a spectroscopic tag and a layer of an antibody that controls recognition specificity. Previous work resulted in the detection of only ˜60 binding events using 30-nm ERLs, which translated to a limit of detection of ˜30 fM in an assay for prostate specific antigen in human serum. The inventors more recently reported on the detection of single-digit binding events via larger (60 nm) ERLs, (Park, H.-Y.; Lipert, R. J.; Porter, M. D. Proc. SPIE 2004; 464-477), which optimized plasmon coupling with the underlying gold substrate at the laser excitation wavelength. (Park, H.-Y.; Lipert, R. J.; Porter, M. D. Proc. SPIE 2004; 464-477; and Driskell, J. D.; Lipert, R. J.; Porter, M. D. J. Phys. Chem. B 2006, 110, 17444-17451).

While proving extremely sensitive, there are several challenges to advancing the scope of this readout strategy. One major obstacle rests with the long incubation times required by both the capture and labeling steps when under diffusion control. This complication is amplified by our assay format because the larger size of ERLs translates to lower diffusional mass transfer rates than those of more typical labels (e.g., fluorescently tagged antibodies). Estimates, which are based only on consideration of particle size via the Stokes-Einstein equation, (Berry, R. S.; Rice, S. A.; Ross, J. Physical Chemistry; John Wiley & Sons: New York, 1980) yield a diffusion coefficient for a 60-nm ERL that is roughly tenfold smaller than that of a fluorescently tagged antibody. Equation 1 therefore indicates that the labeling step with ERLs will be about three times slower than that for a fluorescently tagged antibody. By capitalizing on the second term in Equation 1, it should be possible to use substrate rotation to overcome the diffusion-based limitations to mass transfer in both the capture and labeling steps.

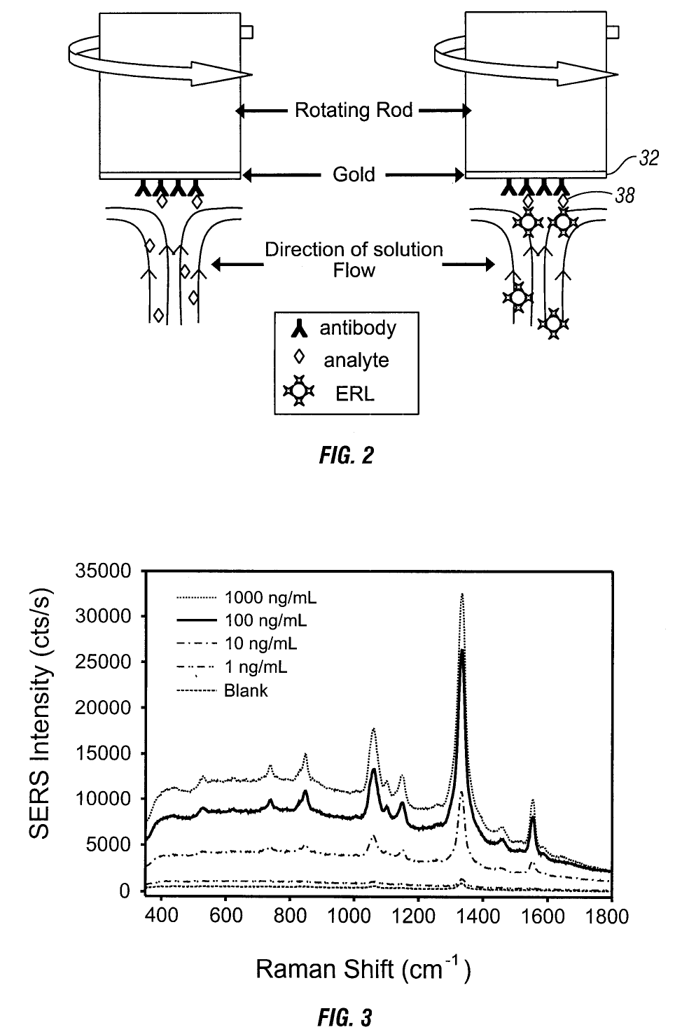

The two steps of the assay are conceptualized in FIG. 2. One end of a rotating rod 24 is coated with gold 26 and modified with dithiobis (succinimidyl propionate) (DSP) 28. Next, an antibody 30, which in this work is anti-rabbit IgG, is coupled to the DSP-modified surface via succinimidyl ester chemistry, with the resulting capture substrate 32 lowered into the sample 34. The rod 24 is then rotated at a controlled rate, which extracts rabbit IgG 36 onto the capture substrate 38. After rinsing, the capture substrate 38 is immersed in an ERL solution 40 and again rotated at a controlled rate. This step labels the captured antigen 38 for detection, which is then subsequently quantified by the spectral intensity of the Raman scatterer on the ERL.

In the example reported below we show that: 1) the ERL labeling step, like the antigen capture step, can be accelerated via substrate rotation; and 2) rotation can be used in assays in a complex biological matrix (i.e., goat serum). Speed of rotation, as well as time of rotation will vary depending upon the assay and the materials and instrument used. Generally the speed should not be so fast as to cause vorterxing or damage to the assay substrate. In the examples here 800 or 1200 rpms for 10 to 15 minutes were sufficient.

The following described example offers an illustration of and demonstrates the effectiveness of the invention in the context Raman's spectroscopy. It should however be understood that this is offered for non-limiting illustrative purposes only. In that sense it is exemplary of one of the many methodologies whose process time may be significantly reduced by use of the improved rotation technique during antigen sample incubation and detection label incubation.

### EXAMPLE

Gold nanoparticles [60-nm diameter (<8% variation), 2.6×10¹⁰ particles/mL] were purchased. Octadecanethiol (ODT), DSP, and phosphate buffered saline (PBS) packs (10 mM, pH 7.2) were obtained from Sigma. SuperBlock and BupH Borate Buffer Packs (50 mM, pH 8.5) were acquired from Pierce. DSNB [5,5′-dithiobis(succinimidyl-2-nitrobenzoate)] was synthesized. All buffers were passed through a 0.22-μm syringe filter (Costar). Contrad 70 (Decon Labs), a mild detergent, was used to clean the glass substrates. Poly(dimethyl siloxane) (PDMS, Dow Corning) was used to prepare microcontact printing stamps.

Goat anti-rabbit IgG polyclonal antibody was purchased from US Biological. The antibody was purified by immunoaffinity chromatography, and supplied as 0.5 mg/mL in PBS (pH 7.2) containing 0.01% sodium azide and 40% glycerol. Experiments show that the performance of the assay varied slightly with each batch of the antibody, and an approach to account for this variation is detailed later. Whole molecule rabbit IgG, also acquired from US Biological, was purified by Protein A affinity chromatography and stored at 10 mg/mL in PBS (pH 7.2). Unless noted, rabbit IgG was diluted with 10 mM PBS. Normal goat serum was obtained from Pierce and acted as a biological matrix for rabbit IgG dilution. This serum (pH 7.2) has a protein concentration of 60 mg/mL.

ERLs are designed to provide a strong Raman signal and selective recognition by, in this case, immunospecificity. As such, DSNB was chosen as the Raman reporter molecule because of the intrinsically strong Raman scattering cross section of its symmetric nitro stretch, the ability of its disulfide moiety to chemisorb to gold surfaces, and the capacity of its succinimidyl ester to covalently conjugate antibodies. This design minimizes the separation of the Raman scatterer and the nanoparticle, yielding a large surface enhancement. This component of the design reflects recent reports that enhancements undergo a sharp decrease (d⁻¹²) as the distance (d) between the particle surface and scattering mode increases.

The ERLs are constructed by first adjusting the pH of a 1.0-mL suspension of 60-nm gold to pH 8.5 via 40.0 μL of 50 mM borate buffer. This pH deprotonates the amines of the antibody added in subsequent steps, which promotes the reaction with the succinimidyl ester of DSNB and stabilizes the suspension after antibody conjugation. Next, 10.0 μL of 1.0-mM DSNB, dissolved in acetonitrile, was pipetted into the suspension and mixed for ˜12 h to form a DSNB-derived layer on the gold particles. This step was followed by the addition of 20 μg of antibody (40.0 μL at 0.5 mg/mL), with the resulting suspension reacted for ˜8 h. As detailed earlier,³⁰ this amount of antibody fully coats the nanoparticles and maintains a stable suspension upon the addition of salt.

To block any unreacted succinimidyl ester groups, 100 μL of 10% BSA in 2 mM borate buffer was added to the particle solution and reacted for ˜12 h. The suspension was then centrifuged at 2000 g for 10 min to remove excess DSNB, antibody, and other residual materials. After decanting the supernatant, the nanoparticles were resuspended in 1.0 mL of 2 mM borate buffer containing 1% BSA. This cleanup cycle was carried out three times. Next, to mimic physiological conditions, concentrated NaCl was added to the ERLs to yield a final salt concentration of 150 mM, and the volume was adjusted to give a nanoparticle concentration of 5.2×10¹⁰ particles/mL. Lastly, the suspension was passed through a 0.22-μm syringe filter to remove any large aggregates.

Next the capture substrate was prepared. Glass microscope slides (Fisher), cut into 1×1 cm squares, were ultrasonically bathed in 10% Contrad 70, deionized water, and ethanol, each for 30 min. The squares were then dried and coated with 15 nm of chromium and 250 nm of gold by resistive evaporation (Edwards 306A evaporator), both at a rate of 0.1 nm/s and pressure less than 7.5×10⁻⁷ Torr. Upon removal from the evaporator, each substrate was addressed by ˜30-s exposure to an ODT-saturated PDMS stamp that had a 4.0-mm hole cut in its center. Next, the substrates were rinsed with ethanol and dried with a stream of high-purity nitrogen. This stamping procedure forms a hydrophobic, ring-shaped barrier on the outer portion of the substrate, defining a sample address that localizes reagents in the center of the substrate and minimizes sample and label consumption. The substrates were subsequently immersed in a 0.1 mM ethanolic solution of DSP for 8 h to form a DSP-derived monolayer on the uncoated portion of the substrate. Finally, the substrates were removed from the DSP solution, rinsed with ethanol, and dried with a stream of high-purity nitrogen.

The capture substrates were completed by pipetting 20.0 μL of 100 μg/mL anti-rabbit IgG (diluted in 50 mM borate buffer) onto the DSP-modified domains of the gold substrates. After allowing 8 h for antibody coupling, the substrates were rinsed three times with 2 mL of 10 mM PBS. Lastly, 20.0 μL of SuperBlock blocking buffer were placed on the capture substrate for 12 h to block any unreacted succinimidyl groups, with the capture substrates then rinsed with 10 mM PBS.

The immunoassay protocol was as follows. The overall goal of these experiments is the demonstration that assay times can be lowered to 30 min or less via substrate rotation. The majority of this time can be devoted to incubation since the SERS readout of the assay requires only 1 s for signal integration. Thus, incubation times of 10 and 15 min were tested under rotation conditions. The capture substrates were exposed to sample solutions (PBS or goat serum) containing varied levels of rabbit IgG. The assays performed under stagnant conditions (i.e., no rotation) exposed 20.0 μL of sample to the capture substrate for 10 min or 12 h in a humidity chamber. Assays preformed under rotation, however, required 1.5 mL of sample to fully submerge the substrate.

After incubation, all samples were rinsed three times in 2 mL of 2 mM borate buffer (pH 8.5) containing 1% BSA and 150 mM NaCl. As subsequently specified, the capture substrates were then exposed to 20.0 μL of ERLs for 10 min or 12 h without rotation, or to 1.5 mL of ERLs for 10 or 15 min with rotation at either 800 or 1200 rpm. With the current apparatus, rotation at 1200 rpm occasionally resulted in undesirable solution vortexing. Therefore, 800 rpm was used in most experiments. After incubation, the samples were rinsed with the aforementioned borate buffer and dried under a stream of high-purity nitrogen. The SERS spectra were then collected.

Capture substrates were attached to the end of a 17-cm long, stainless steel rod (6-mm diameter) by double-sided tape (3M). The rod readily mates with an AFMSRX rotator (Pine Instrument Company). The substrate was then lowered into a sample or labeling well (17-mm diameter) and rotated at a controlled rate with the AFMSRX analytical rotator. The rotator has an accuracy of 1% between 0 and 10,000 rpm. The slew rate of the motor is 300,000 rpm/s; therefore, the desired rotation rate was effectively attained instantaneously for the rotation rates (800 or 1200 rpm) and incubation times (10 or 15 min) used herein.

Raman spectra were collected with a NanoRaman I (Concurrent Analytical) fiber-optic Raman system. The excitation source is a 30-mW, 632.8-nm HeNe laser. The spectrograph consists of an f/2.0 Czerny-Turner imaging spectrometer (resolution of 6-8 cm⁻¹) and a thermoelectrically cooled (0° C.) CCD (Kodak 0401E). The probe objective (numerical aperture 0.68) focuses the laser to a 25-μm diameter spot on the substrate surface; it also collects the scattered Raman radiation. All spectra were acquired with a 1-s integration time.

As a starting point, experiments to detect rabbit IgG were performed by following our earlier protocol (Driskell, J. D.; Kwarta, K. M.; Lipert, R. J.; Porter, M. D.; Neill, J. D.; Ridpath, J. F. Anal Chem. 2005, 77, 6147-6154). This approach served both as a comparative standard and as a control in order to account for differences in performance due to variations between batches of vendor-supplied antibodies. The protocol calls for a 12-h incubation of the capture substrate with 20.0-μL samples of rabbit IgG, followed by a 12-h incubation in 20.0 μL of ERLs. Both steps are performed in stagnant solution. We add that our studies (data not shown) demonstrated that anti-rabbit IgG has excellent specificity when used in the construction of both the capture substrate and ERLs. That is, in tests with human, rat, mouse, and rabbit IgGs, only exposures to rabbit IgG yielded signals that exceeded those of blank solutions.

The Raman spectra for a blank solution and for varied concentrations of rabbit IgG are presented in FIG. 3. These spectra contain features characteristic of the DSNB-based Raman reporter molecule, confirming the presence of the DSNB-modified ERLs. The dominant feature in the spectra is the symmetric nitro stretch [vₛ(NO₂)] at 1336 cm⁻¹, which will be used for antigen quantification. Other prominent features include the nitro scissoring vibration at 851 cm⁻¹, an aromatic ring mode at 1566 cm⁻¹, and a succinimidyl N—C—O stretch that overlaps with other aromatic ring modes at 1079 cm⁻¹.

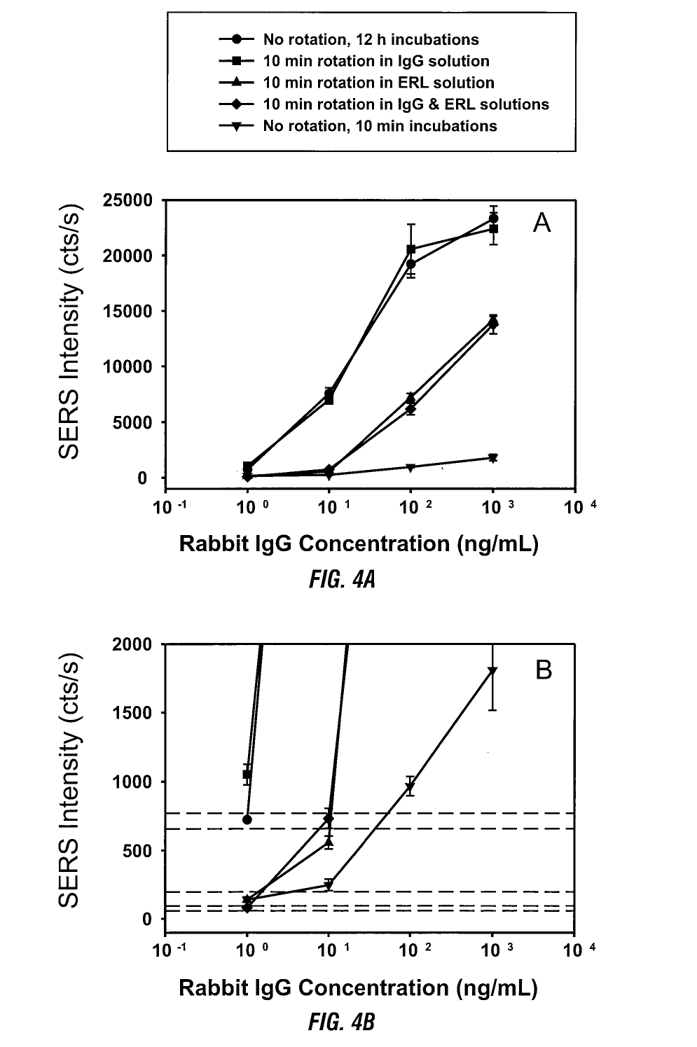

FIG. 3 shows that all spectral intensities vary proportionally with rabbit IgG concentration. This dependence is given by a plot of the intensity of vₛ(NO₂) versus rabbit IgG concentration, which is presented in FIG. 4A and is labeled “No rotation, 12 h incubations.” Each data point is the mean intensity of vₛ(NO₂) from five locations on a single sample and the error bars represent the standard deviation in the signal. Sample-to-sample variations were under 10%. FIG. 3 also indicates that the signal due to the nonspecific adsorption of ERLs in the blank approaches that of the 1-ng/mL sample. Formally, the limit of detection, defined as the concentration that results in a signal equal to that of the blank plus three times its standard deviation and calculated by using the slope of the response between 1 and 10 ng/mL, is 1 ng/mL.

To document the importance of incubation time in a quiet solution, an immunoassay for rabbit IgG was performed under static conditions by exposing the substrate to the sample and ERL solutions for 10 min each. Although not shown, the spectral intensities are much weaker than those obtained for the 12-h incubations. The resulting dose-response curve is also shown in FIG. 4A and is labeled “No rotation, 10 min incubations.” While an antigen concentration dependent response is again observed, the sensitivity is markedly decreased compared to the assay with 12-h incubations. The signal, for example, for 10 ng/mL rabbit IgG is 1/30th that for the corresponding 12-h incubations. Interestingly, the signal for nonspecific binding is also significantly reduced (˜5×, see FIG. 4B) for the short incubation. Thus, by using the slope between 10 and 100 ng/mL, there is only a fourfold degradation in the detection limit to 4 ng/mL with the 10-min binding times. Collectively, these data emphasize the importance of incubation time and suggest that increasing antigen and label flux to the sample surface could lead to large improvements in assay performance.

The influence of substrate rotation on antigen and ERL binding was investigated to assess its potential to shorten the time required for the immunoassay and lower the limit of detection. First, an immunoassay was performed in which the capture substrate was rotated at 800 rpm for 10 min in the sample solution. Based on Equation 1, these conditions should result in a fivefold increase in antigen impingement compared to that for stagnant binding for 12 h. After extraction, the samples were exposed to a quiet solution of ERLs for 12 h. The dose-response curve for this assay is shown in FIG. 3A, labeled “10 min rotation in IgG solution.” Interestingly, this curve is strongly similar to that for the 12-h stagnant incubation. The absence of the expected increase in binding with rotation suggests that in both cases an equilibrium between free and surface-bound IgG is being approached and therefore the assumptions underlying Equation 1 are no longer applicable. This situation is in stark contrast to the strong agreement between Equation 1 and the results for virus binding found earlier. We ascribe the difference in the two studies to the ˜6× larger diffusion coefficient of the IgG proteins used herein with respect to that for the viruses used earlier.

FIG. 4B also shows a small decrease in nonspecific binding. The detection limit was thus calculated to be 0.4 ng/mL, based on the slope of the plot from 1 to 10 ng/mL. These results demonstrate, as detailed earlier, that rotation-induced flux is a highly effective means of antigen delivery in that the time required for an assay can be reduced without a loss in the limit of detection.

Two studies investigated the effect of substrate rotation on ERL binding. In one, rabbit IgG was exposed to the capture substrate for 12 h in quiet solution prior to capture substrate rotation at 800 rpm for 10 min in an ERL solution (labeled “10 min rotation in ERL solutions” in FIG. 3). In the other, a capture substrate was rotated at 800 rpm for 10 min in both the antigen solution and then in ERLs (labeled “10 min rotation in IgG & ERL solutions”). It is readily apparent that the dose-response curves obtained in the two experiments are strongly comparable (FIG. 4A).

Several important conclusions can be drawn from these experiments. First, as expected, equivalent dose-response curves can be constructed with substrate rotation in the labeling solution using either approach for capturing antigen. This provides further support for the conclusion drawn from the first set of experiments with respect to the approach to equilibrium for the binding of antigen to the capture surface. The second noteworthy observation from FIG. 4A is that smaller signals are obtained when ERLs bind under rotation, in contrast to the results of rotation during antigen binding. Moreover, first approximations via Equation 1 estimate the ERL impingement to be three times greater than ERL labeling in quiet solution. We therefore expected the signals would be the same or larger than those measured using stagnant ERL incubation. Importantly, the nonspecific binding of the ERLs, shown in FIG. 4B, is significantly lower for the assays utilizing rotation to bind the label. Consequently, a detection limit of ˜1 ng/mL was also obtained for these assays.

There are several possible origins for the decrease in specific ERL binding under rotation. One possibility is that rotation in ERL solution, which is initially antigen-free, drives the equilibration of the system more rapidly with respect to the loss of captured antigen. A second potential reason is that rotation does not deliver as many ERLs to the surface as realized via diffusion for 12 h, contrary to first projections. If so, the value of D for the ERLs, which was approximated by changing the particle radius in the Stokes-Einstein equation, must be lower than initially estimated. However, the value for D of the ERLs necessary to produce the observed drop in signal is unrealistically small and can be, at best, only partially responsible. Another possibility for the unexpected drop in signal with rotation in ERLs is that the reaction of ERLs with captured antigen is not diffusion limited but is limited by the rate of reaction between ERL and antigen.

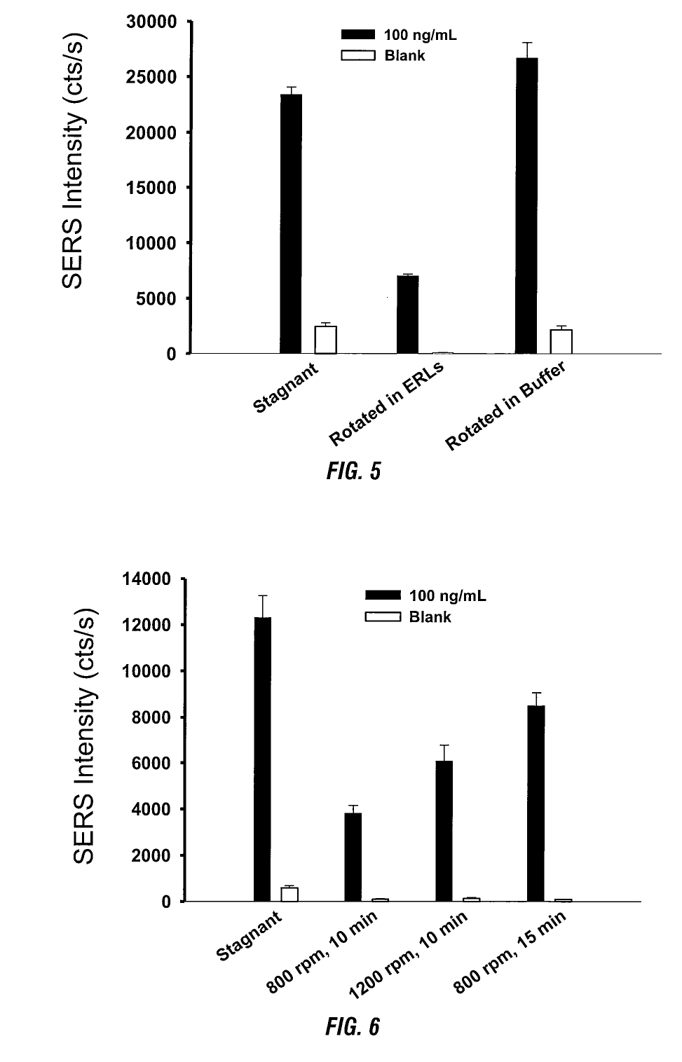

Tests were performed to determine if rotation during ERL labeling caused the loss of bound antigen. For this, several capture substrates were prepared and exposed to 100 ng/mL rabbit IgG (20.0 μL) for 12 h. As controls, one substrate was exposed to 20.0 μL of stagnant ERLs for 12 h, while another was rotated at 800 rpm for 10 min in ERLs. A third substrate was spun in 2 mM borate buffer (1% BSA, 150 mM NaCl) at 800 rpm for 10 min in order to assess the impact of rotation in solution devoid of ERLs. This sample was then exposed to 20 μL of ERLs without rotation. Blank studies were also performed under each of these conditions. The resulting SERS signals are shown in FIG. 5.

The signal obtained for the substrate rotated in buffer prior to labeling with ERLs under static conditions was similar to that for the sample labeled without rotation. As before, the substrate exposed to ERLs with rotation gave a weaker signal. These data show that the solution flow induced by rotation step does not affect the amount of bound antigen, indicating that there must be another mechanism that gives rise to the lower signal observed when labeling is performed with substrate rotation. It is also important to note that the blank signals in these assays are consistent with previous results: there is much less nonspecific binding when labeling with rotation.

The rotation rate was increased and the incubation time was lengthened to increase the surface coverage of bound ERLs, as predicted by Equation 1. Substrates exposed to 100 ng/mL rabbit IgG (20 μL) for 12 h were then incubated with ERLs for 12 h under stagnant conditions, or rotated in an ERL solution for 10 min at 800 rpm, 10 min at 1200 rpm, or 15 min at 800 rpm. Control substrates were exposed to 10 mM PBS in place of the rabbit IgG and then incubated with ERLs under the conditions outlined above. The measured intensities of vₛ(NO₂) for each substrate are plotted in FIG. 6. While this set of experiments was performed with a new batch of antibodies and the signal for the 100 ng/mL control (i.e., stagnant incubation) is lower than that obtained in earlier studies, it is evident that the signal obtained for the 100 ng/mL sample of IgG increases as the rotation rate increases from 800 to 1200 rpm and as the incubation time increases from 10 to 15 min. The discrepancy in signal from earlier studies is attributed to differences in antibody performance.

Analysis of these results supports the hypothesis that ERL impingement, and hence labeling, qualitatively follows Equation 1. First, rotation-induced flux, and therefore Γₐ, is directly proportional to time, but only to the square root of rotation rate. Thus, the signal is expected to increase more for a 50% increase in incubation time compared to a 50% increase in rotation rate. This expectation was experimentally realized. However, the ERL solution vortexes at 1200 rpm, which precludes a more exacting analysis since the theory for rotation-induced flux applies only to laminar flow profiles. Nevertheless, the increase in signal with increased incubation time and rotation rate demonstrates that the system has not reached equilibrium under these conditions.

To this point, the experiments have verified that: 1) the labeling step does not remove antigen; 2) equilibrium has not been reached in the labeling step; and 3) the D-value of the ERLs is not solely responsible for the decreased signals when labeling with rotation. The combined weight of these results therefore suggests that Equation 1 may not a quantitatively reliable model for the labeling step. As noted in the introduction, there are three assumptions central to the derivation of this equation; all apply to the antigen capture and ERL labeling steps. First, the reactant solution concentration is independent of binding. Second, the binding sites at the surface do not saturate. Third, the recognition reaction is fast compared to the delivery of reactant. The first and second assumptions are likely valid in some of the experiments but not in others. It is also not known if the third assumption is applicable to ERL labeling. More experiments are needed to gain further insights into each the processes involved. Nevertheless, the results suggest that signal strengths achieved without rotation can be realized by increasing ERL impingement via rotation.

Per Equation 1, signal increases can be realized by further increasing the rotation rate, the incubation time, the ERL concentration, or any combination of the three. Moreover, the blank signal in FIG. 5 shows that the level of nonspecific binding, irrespective of rotation rate or incubation time, remains lower than with stagnant incubation. Therefore, it should be possible to lower the limit of detection with rotation compared to the control assay while reducing the assay time from ˜24 h to ˜30 min, in excess of a 95% reduction!

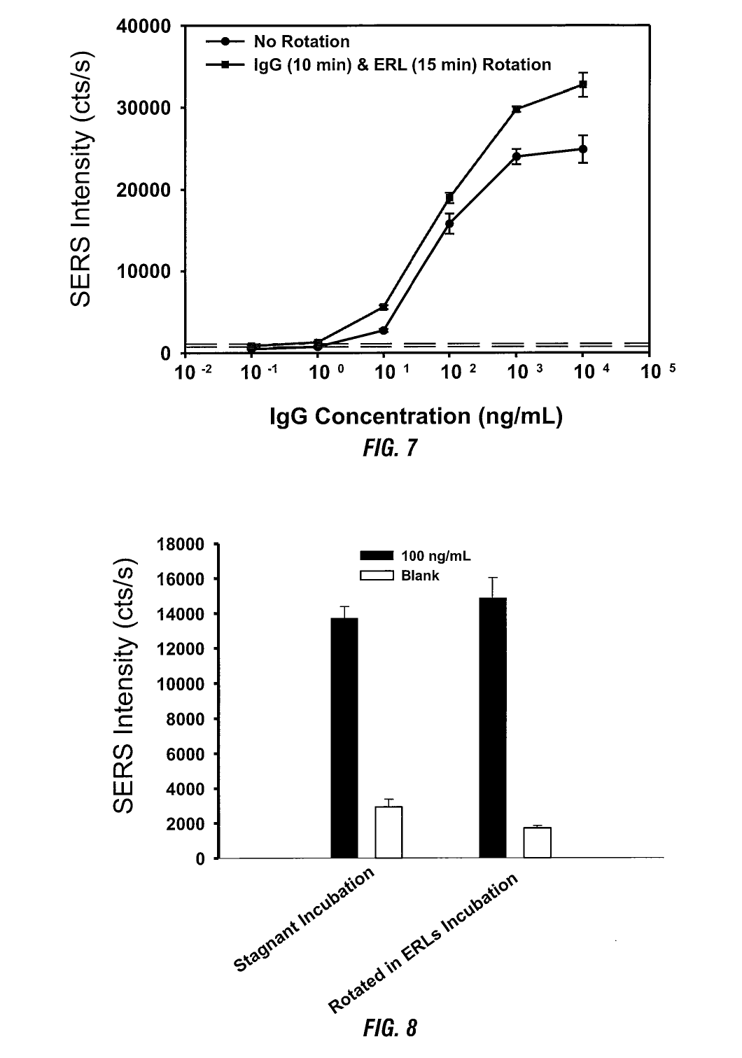

An optimized assay was performed by identifying an appropriate rotation rate, incubation time, and ERL concentration and evaluated against a control assay. A rotation rate of 800 rpm was selected to maintain laminar conditions and the incubation time held at 15 min. Larger signals could be realized with a longer incubation time; however, in light of the overall goal of decreasing the assay time, other means of obtaining signal equivalent to the control assay are preferred. Therefore, the concentration of ERLs was increased from 5.2×10¹⁰ ERLs/mL, the concentration used in the control assays, to 10.4×10¹⁰ ERLs/mL in an effort to increase the overall ERL impingement. The results of this assay, and those of a control assay, are shown in FIG. 7.

There are several noteworthy observations from the two curves. First, larger signals are obtained for substrates rotated in the ERL labeling solution. These larger signals are due to the doubling of ERL concentration. This can be seen by noting the signal at 100 ng/mL is approximately double the signal shown in FIG. 6 for the same rotation conditions but half the ERL concentration. Also, less nonspecific binding occurs for the substrates that are rotated in the ERL solution. This results in a detection limit of ˜1 ng/mL for the assay performed with rotation compared to ˜10 ng/mL for the assay without rotation. It can be seen from the detection limit for the control assay that detection limits varied for each batch of antibody received from the vendor, but this tenfold improvement in detection limit was consistently observed.

Detection of a protein in PBS is only realistic if the sample has been heavily purified. An assay would preferably be performed, for example, directly on a blood serum sample, which would potentially contain high levels of nontargeted proteins that could degrade performance via nonspecific adsorption. To this end, an assay was performed for rabbit IgG suspended in goat serum. Following the standard protocol for a stagnant assay, control substrates were exposed to either 20.0 μL of 100 ng/mL rabbit IgG diluted in goat serum or 20.0 μL of blank goat serum for 12 h, followed by incubation with 20.0 μL of ERLs (5.2×10¹⁰ ERLs/mL) for 12 h. Separate capture substrates were also rotated at 800 rpm for 10 min in the serum-based sample and blank solutions and then at 800 rpm for 15 min in ERLs (10.4×10¹⁰ ERLs/mL).

The results are shown in FIG. 8, and indicate that similar signals were obtained for spiked goat serum and spiked PBS. Like the assay in PBS (FIG. 7), the signal for the rotated substrate in spiked serum (FIG. 8) is slightly larger than that for the statically incubated substrate. Moreover, the level of nonspecific binding is again found to be less for the rotated sample. The serum blank, however, yields a larger amount of nonspecific binding than the PBS blank, which results in a detection limit for rabbit IgG in a serum matrix with and without rotation of ˜10 and ˜30 ng/mL, respectively. While preliminary in that more effort could be placed on finding a more effective blocking agent, these data demonstrate that substrate rotation can be successfully applied to real sample matrices for the reduction of assay time and lowering of detection limit.

We conclude by applying rotation to an assay for rabbit IgG from goat serum; this study, when compared to the assay performed under static conditions, demonstrates that the time for the assay can be reduced from several hours to ˜25 min, and that this reduction is accompanied by a tenfold improvement in detection limit.

This improved process as illustrated by the Example is the first report on the combination of rotation-induced flux and SERS readout in a sandwich-type immunoassay format. Systematic studies of the influence of rotation on antigen and label binding led to an optimized immunoassay yielding a tenfold decrease in the limit of detection (i.e., ˜10 ng/mL to ˜1 ng/mL) and a reduction in the assay time from 24 h to 25 min compared to a static immunoassay. Additionally, rotation-induced flux was effectively applied to samples in a serum matrix. We found that labeling under convective conditions reduces nonspecific binding, the factor responsible for restrictions on the lowest level of detection. We are beginning further investigation into the mechanism of nonspecific binding. Insights into the role of rotation rate, incubation time, and label concentration on nonspecific binding have the potential to significantly improve the limit of detection for immunoassays employing a wide variety of readout techniques.

As can be seen from the above foregoing, the invention accomplishes the primary objective set forth in the initial description.

1. In the process of performing heterogeneous immunoassays involving a period of substrate incubation time to capture antigens and/or label samples, the improvement comprising:
rotating the substrate during incubation time at a speed sufficient to reduce incubation time for from 50% to 90%, but below the speed at which vortexing occurs for antigen capture or sample labeling time to reduce the time of antigen capture and/or sample labeling.

2. The process of claim 1 wherein the heterogeneous immunoassays is selected from the group consisting of scintillation counting, fluorescence, chemiluminescence, electrochemical assays and enzymatic methods, surface plasmon resonance, surface-enhanced Raman scattering, quantum dots, and microcantilevers.

3. The process of claim 2 wherein the immunoassays is surface enhanced Raman scattering.

4. (canceled)

5. The process of claim 1 wherein the total incubation time is reduced 95%.

6. (canceled)

7. The process of claim 1 wherein rotation occurs during both the time of antigen capture and sample labeling.

8. The process of claim 7 wherein the heterogeneous immunoassays is selected from the group consisting of scintillation counting, fluorescence, chemiluminescence, electrochemical assays and enzymatic methods, surface plasmon resonance, surface-enhanced Raman scattering, quantum dots, and microcantilevers.

9. The process of claim 8 wherein the immunoassays is surface enhanced Raman scattering.

10. (canceled)

11. The process of claim 7 wherein the total incubation time is reduced 95% of the stagnant times.

12. (canceled)

13. A method of reducing assay time for heterogeneous immunoassays prepared by using an antigen binding step and thereafter a labeling step for the antigen/antibody substrate mix, comprising:
rotating the substrate during incubation time for an antigen/antibody mixture and during the labeling step;
at a speed sufficient to reduce the incubation time from 50% to 90% of that time needed without rotation for formation of the antigen/antibody substrate mix.
